(19) United States
(12) Patent Application Publication
(10) Pub. No.: US 2026/0007324 A1
(43) Pub. Date: Jan. 8, 2026

(54) SYSTEM AND METHOD FOR LOCATING A MEDICAL DEVICE USING AN ELECTRICAL FIELD CREATION

(71) Applicant: Anumana, Inc., Cambridge, MA (US)

(72) Inventors: Deepak Anand, Doddanekundi (IN); Rakesh Barve, Bengaluru (IN); Rohit Jain, Danville, CA (US); Animesh Agarwal, San Francisco, CA (US); Pankaj Lakhwan, Bengaluru (IN)

(73) Assignee: Anumana, Inc., Cambridge, MA (US)

(21) Appl. No.: 18/764,853

(22) Filed: Jul. 5, 2024

(51) Int. Cl. A61B 5/06; A61B 5/00; A61B 5/287

(52) U.S. Cl. CPC A61B 5/063; A61B 5/287; A61B 5/6859; A61B 2560/0223; A61B 2560/0468; A61B 2562/046

(57) ABSTRACT

A system for locating a medical device using an electrical field creation comprising a plurality of excitation patches positioned on a hemispherical electrical field, a common ground patch, at least a catheter assembly comprising at least a tip comprising a plurality of electrodes, and a processor configured to transmit an electric signal between each excitation patch and the common ground patch; measure at least a voltage at the plurality of electrodes; generate an assessment of an impedance location an electrode of the plurality of electrodes as a function of the plurality of voltages; and retrieve a location of the device using of the at least a voltage and the assessment of the impedance location.

### FIELD OF THE INVENTION

The present invention generally relates to the field of electrophysiological mapping. In particular, the present invention is directed to locating a medical device using an electrical field creation.

### BACKGROUND

Conventional methods of excitation in electrophysiological mapping involve using paired patches with varying field strengths. This variation necessitates compensating for weaker field strengths and dead zones which can result in inaccurate readings.

### SUMMARY OF THE DISCLOSURE

In an aspect a system for locating a medical device using an electrical field creation. The system may include a plurality of excitation patches positioned on a hemispherical electrical field wherein the plurality of excitation patches references a common ground patch, wherein each excitation patch in the plurality of excitation patches includes an individual frequency; at least a catheter assembly comprising at least a tip, wherein the tip is comprised of a plurality of electrodes; at least a processor communicatively connected to the plurality of excitation patches and the at least a catheter assembly; a memory communicatively connected to the at least a processor, wherein the memory contains instructions configuring the at least a processor to transmit an electric signal voltage between each excitation patch of the plurality of excitation patches and the common ground patch; measure at least a voltage at the plurality of electrodes; generate an assessment of the an impedance location an electrode of the plurality of electrodes between each excitation patch of the plurality of excitation patch and the plurality of electrodes as a function of the plurality of voltages; and retrieve a location of the medical device as a function of the measured at least a voltage at the plurality of electrodes and the assessment of the impedance location.

In another aspect a method for locating a medical device using an electrical field creation. The method may include transmitting an electric signal between each excitation patch of the plurality of excitation patches and the common ground patch, measuring at least a voltage at the plurality of electrodes, generating an assessment of an impedance location at an electrode of the plurality of electrodes as a function of the plurality of voltages; and retrieving a location of the medical device as a function of the at least a voltage at the plurality of electrodes and the assessment of the impedance location.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a flow diagram illustrating a system for locating a medical device using an electrical field creation;

FIG. 2A-B are diagrams of exemplary positionings of the plurality of excitation patches on a human body;

FIG. 3 is a diagram of an exemplary positioning of the plurality of excitation patches on a three-dimensional volume;

FIG. 4 is a diagram of an exemplary embodiment of a hemispherical field within a three-dimensional volume;

FIG. 5 is a diagram of an exemplary embodiment of a catheter assembly;

FIG. 6 illustrates a block diagram of an exemplary embodiment of a machine learning module;

FIG. 7 illustrates a diagram of an exemplary nodal network;

FIG. 8 illustrates a block diagram of an exemplary node;

FIG. 9 illustrates an exemplary embodiment of a cylindrical field within a three-dimensional volume;

FIG. 10 illustrates a method for locating a medical device using an electrical field creation; and

FIG. 11 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for locating a medical device using an electrical field creation. In an embodiment, a hemispherical electrical field is created using a plurality of excitement patches strategically positioned within a three-dimensional volume.

Aspects of the present disclosure can be used to determine a location of a medical device using a voltage measurement and an assessment of an impedance location.

Aspects of the present disclosure allow for locating a medical device using an electrical field creation. Exemplary embodiments illustrating aspects of the present disclosure are described below in the context of several specific examples.

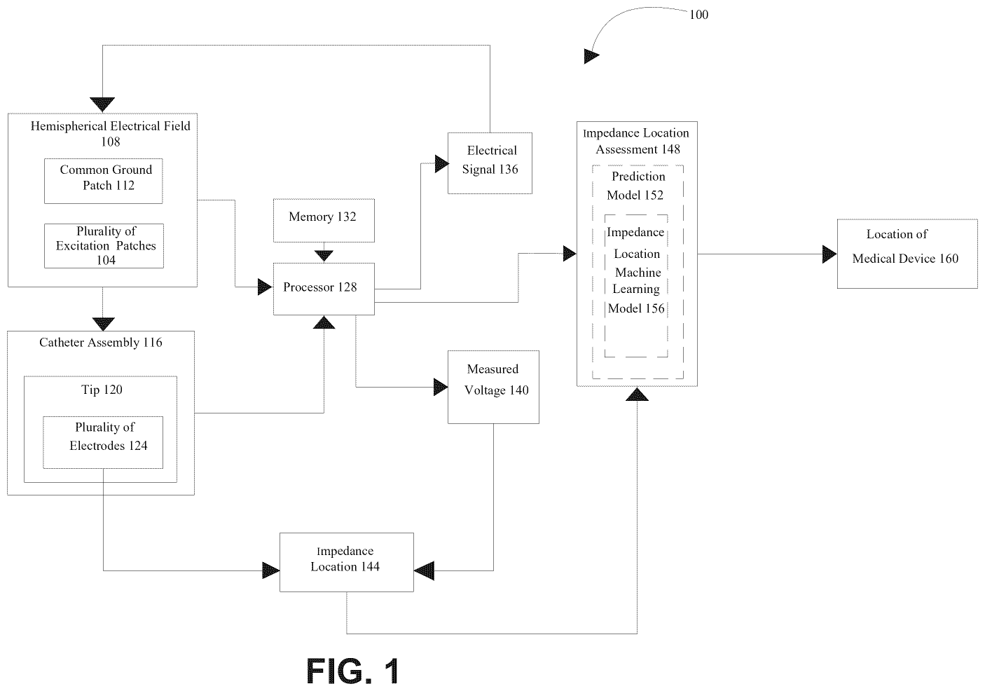

Referring now to FIG. 1, an exemplary embodiment of a system 100 for locating a medical device using an electrical field creation is illustrated. As used in this disclosure, a “medical device” is an instrument, apparatus, implement, machine, implant, and the like used to perform or aid in performing a medical procedure. In a non-limiting embodiment, a medical device may include a catheter, gastroscope, colonoscope, dialysis sheath, coronary stent, and the like.

Continuing reference to FIG. 1, system 100 may include a plurality of excitation patches 104 configured to generate a hemispherical electrical field 108. As used in this disclosure, an “excitation patch” is a patch equipped with electrodes that deliver electrical signals. In some embodiments, the excitation patch is configured to stimulate and record a cardiac tissue's electrical response, which is then may be used to create an electrophysiological (EP) map. As used herein, an “electrophysiological map” is a detailed representation of the electrical activity within the heart. The EP map may be created using data collected from electrodes placed on or inside the heart. An EP map may be used to allow visualization of timing and pathways of electrical signals to identify areas of abnormal electrical activity. In an embodiment, excitation patch may include at least an electrode arranged in a specific pattern to maximize contact with an area of interest. In another non-limiting embodiment, there may be at least five excitation patches within the plurality of excitation patches. In a non-limiting embodiment, plurality of excitation patches 104 may deliver controlled electrical pulses to cardiac tissue which may be designed to provoke a cardiac electrical activity. In an embodiment, input voltages measuring 50 mV, 50 mV to 75 mV, or 75 mV to 100 mV, may be applied to the plurality of excitation patches. In some embodiments, input voltages may be from 20 mV to 150 mV. In some embodiments, input voltages may be 30 mV to 100 mV. In some embodiments, input voltages may be 40 mV to 60 mV. Plurality of excitation patches 104 may be configured to adjust the intensity, duration, frequency, and the like of the electrical pulses. Each excitation patch within the plurality of excitation patches may comprise an individual frequency. In a non-limiting embodiment, the plurality of excitation patches may reference a common ground patch 112. This configuration may not enforce the orthogonality constraint as processed in conventional pair patch placement methods. In a non-limiting embodiment, the plurality of excitation patches 104 may be placed on three-dimensional volume, wherein the plurality of excitation patches is placed significantly higher near the top of the three-dimensional volume. In another non-limiting embodiment, the plurality of excitation patches 104 may be placed on a hemispherical electrical field. As used herein a “hemispherical electrical field” is a region of electrical activity that is shaped to reflect a half-sphere. The hemispherical electrical field may allow for uniform distribution of an electrical field which may enhance the accuracy for detecting and mapping electrical signals during an EP mapping process. As used herein an “electrical field” refers to the distribution of electrical forces or potentials in a given area, such as the heart. The electrical field may be generated by a flow of electrical current through cardiac tissue. Mapping electrical fields within the cardiac region may allow for the detection of arrhythmias, abnormal electrical pathways, and the like. In an embodiment, the hemispherical electrical field 108 may be configured to ensure that there is no significant dead zone. As used herein, a “dead zone” refers to an area in an EP map where the signal quality is significantly compromised, or the detected signals cannot be reliably detected. A dead zone may occur due to poor electrode contact with cardiac tissue, interference from surrounding structures, and the like. The hemispherical electrical field 108 may prevent the occurrence of a dead zone by utilizing a plurality of excitation patches, each operating at individual frequencies, along with a common ground patch. This configuration creates a hemispherical field that may also avert a weak field scenario. As used herein, a “ground patch” is an electrically conductive area that serves as a reference point for the plurality of excitation patches. The common ground patch may be used to ensure that all measurements pertaining to electrical signals are made relative to a common electrical potential. In a non-limiting embodiment, the positioning of at least an additional excitation patch of the plurality of excitation patches may transform the hemispherical field 108 into a cylindrical field. This transformation from the hemispherical field into the cylindrical field may occur if there are several excitation patches relatively close together at equal spaced distances.

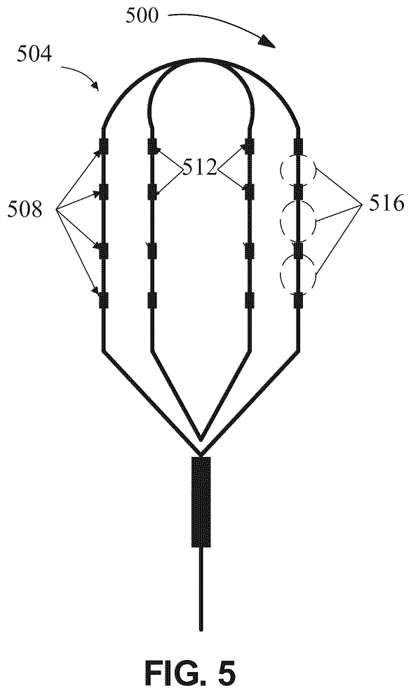

Continuing reference to FIG. 1, system 100 may include at least a catheter assembly 116 comprising at least a tip 120, wherein the tip is comprised of a plurality of electrodes 124. As used herein a “catheter assembly” refers to a flexible, tube-like medical device that is used for various diagnostic and therapeutic procedures within the body. At least a catheter assembly 116 may include at least a tip that is configured to interact directly with body tissues or systems during medical procedures. At least catheter assembly 116 may include a plurality of electrodes 124 on the tip 120. As used in the current disclosure, an “electrode” is a multi-functional component that is used for both diagnostic and therapeutic functions in medical procedures. The electrodes may include conductive components used for therapy, such as delivering targeted treatments like ablation within the body, but they also serve diagnostic purposes. This dual functionality is integral to procedures like cardiac ablation, where precise location and treatment of aberrant electrical pathways are crucial. At least a catheter assembly is further discussed below with reference to FIG. 5.

Continuing reference to FIG. 1, system includes at least a processor 128. At least a processor 128 includes a processor communicatively connected to a memory 132. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1, at least a processor 128 may include any computing device as described in this disclosure, including without limitation a microcontroller, microprocessor, digital signal processor (DSP) and/or system on a chip (SoC) as described in this disclosure. At least a processor 128 may include, be included in, and/or communicate with a mobile device such as a mobile telephone or smartphone. At least a processor 128 may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. At least a processor 128 may include but is not limited to, for example, a computing device or cluster of computing devices in a first location and a second computing device or cluster of computing devices in a second location. At least a processor 128 may include one or more computing devices dedicated to data storage, security, distribution of traffic for load balancing, and the like. At least a processor 128 may distribute one or more computing tasks as described below across a plurality of computing devices of computing device, which may operate in parallel, in series, redundantly, or in any other manner used for distribution of tasks or memory between computing devices. At least a processor 128 may be implemented, as a non-limiting example, using a “shared nothing” architecture.

With continued reference to FIG. 1, at least a processor 128 may be designed and/or configured to perform any method, method step, or sequence of method steps in any embodiment described in this disclosure, in any order and with any degree of repetition. For instance, at least a processor 128 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. At least a processor 128 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, at least a processor 128 may be configured to transmit an electrical signal 136 between each excitation patch of the plurality of excitation patches and the common ground patch. In an embodiment, each excitation patch of the plurality of excitation patches 104 may operate at an individual frequency. In an embodiment, a system 100 may be configured to generate an electrical signal tailored to the requirements of each excitation patch of the plurality of excitation patches. The electrical signal may be transmitted to the excitation patch as a function of a command of processor 128. In some embodiments, a frequency may include 8 kHz. In some embodiments, a first excitation patch 104 may include a first frequency of 6 kHz. In some embodiments, a second excitation patch 104 may include a second frequency of 7 kHz. In some embodiments, a third excitation patch 104 may include a third frequency of 8 kHz. In some embodiments, a fourth excitation patch 104 may include a fourth frequency of 9 kHz. In some embodiments, a fifth excitation patch 104 may include a fifth frequency of 10 kHz. In some embodiments, the frequencies for the plurality of excitation patches may range from 2 kHz to 20 kHz. In some embodiments, the frequencies for the plurality of excitation patches may range from 8 kHz to 10 kHz. In an embodiment, once an excitation patch of the plurality of excitation patches has received an electric signal, the excitation patch may be activated which may cause the excitation patch to emit an electric signal frequency, waveform, or the like. As used herein, an “electric signal” refers to a transfer of electrical energy that varies with time. In an embodiment, after the electrical signal emitted by the intended excitation patch, the electrical signal may be received by common ground patch.

With continued reference to FIG. 1, the process of activating an excitation patch may be repeatedly implemented sequentially to all excitation patches of the plurality of excitation patches, which may ensure consistent activation and operation across the system. This process of sequentially activating the plurality of excitation patches may be referred to as “multiplexing.” Multiplexing may be used to reduce interference between electrical signals. As used herein, “multiplexing” refers to method used to transmit multiple signals over a single communication channel by dividing the time into multiple slots. Each signal may be allocated its own unique time slot within each cycle. This method may ensure that multiple signals can share the same transmission medium without interfering with each other.

In a non-limiting embodiment, the electrical signal transmitted between each excitation patch of the plurality of excitation patches and the common ground patch may be an alternating current. As used herein, an “alternating current” refers to a type of electrical current where the flow of electrons periodically reverses its direction at regular intervals.

With continued reference to FIG. 1, at least a processor 128 may configured to measure at least a voltage 140 at the plurality of electrodes 124. The “at least a voltage” measurement refers to the electrical potential difference between the potential at the electrode relative to the common ground patch 112. Measuring the at least a voltage at the plurality of electrodes 124 may include amplifying or filtering received electrical signals to ensure that only cardiac signals are accurately recorded. Amplifying or filtering the received electrical signals may be conducted to enhance the electrical signal quality and reduce outside noise.

With continued reference to FIG. 1, at least a processor 128 may be configured to generate an assessment 148 of an impedance location 144 of an electrode of the plurality of electrodes as a function of the plurality of voltages. As used herein “impedance location” refers to the position of an electrode or catheter within a three-dimensional volume. Impedance location 144 may be determined by measuring how much an alternating current electrical current and is influenced by both the resistance and reactance of cardiac tissue through which the electrical current may flow through. Measuring an impedance location may be used to ensure accurate placement and navigation of the at least a catheter assembly 116 within the three-dimensional volume. Impedance location 144 may be calculated based on a voltage drop that occurs at an electrical current flow through cardiac tissue. Impedance location 144 may be calculated as a function of the measured voltage 140 at the plurality of electrodes 124.

With continued reference to FIG. 1, in an embodiment, impedance location 144 and/or location of medical device 160 may be calibrated as a function of movement of the three-dimensional volume. Calibration may involve taking into account the movement within the three-dimensional volume, such as movement of the heart, respiratory motion, body movement. Such movements may alter the relationship between the plurality of electrodes and the cardiac tissue. Calibration may utilize motion tracking technologies to track the movement of the three-dimensional object. Data collected from a motion tracking technology may be used to adjust the impedance measurements in real-time or after impedance measurements are taken. For example, during an EP study, respiratory movements within the chest cavity require calibration of the impedance location based on these movements. This ensures that the measurements of electrical activity remain accurate despite the dynamic environment.

With continued reference to FIG. 1, in an embodiment, impedance location calibration may be determined using a calibration machine learning model. Calibration machine learning model may be configured to generate a calibration scaling factor. Calibration scaling factor may be applied to a location measurement, such as impedance location 144 and/or location of medical device 160. This may correct these location measurements for inaccuracies arising from the movement of the three-dimensional volume. In some embodiments, calibration machine learning model may be trained using calibration training data. Calibration training data may include changes in location of plurality of electrodes correlated to calibration scaling factors. A “machine-learning process,” as used in this disclosure, is a process that automatedly uses a body of data known as “training data” and/or a “training set” (described further below in this disclosure) to generate an algorithm that will be performed by at least a processor 128 to produce outputs given data provided as inputs; this is in contrast to a non-machine learning software program where the commands to be executed are determined in advance by a user and written in a programming language. A machine-learning process may utilize supervised, unsupervised, lazy-learning processes and/or neural networks, described further below. Machine learning plays a crucial role in enhancing the function of software for generating an impedance location machine-learning model. This may include identifying patterns within the predicated voltage deviations that lead to changes in the capabilities of the impedance location machine-learning model. By analyzing vast amounts of data related to predicted voltage deviations, machine learning algorithms can identify patterns, correlations, and dependencies that contribute to the generation of the calibration location machine-learning model. In some embodiments, calibration training data may use historical inputs and outputs, outputs from previous iterations of processing, example inputs and outputs, and the like.

With continued reference to FIG. 1, in an embodiment, generating an assessment 148 of an impedance location may include a prediction model 152. Based on the measured voltage at an electrode, the prediction model may be configured to determine a relative distance to an excitation patch. The relative distance to the excitation patch may then be converted to absolute coordinates (e.g. x, y, z) using the known positions of the excitation patch. The prediction model may be configured to convert the voltage deviation to the impedance location for the plurality of electrodes. Prediction model 152 may be configured to use a plurality of relative distances to excitation patches to determine, using geometric mean, the absolute location of the catheter (e.g., using x, y, z, coordinates that are global to the three-dimensional volume. In an embodiment, the voltage deviation may be calculated by comparing recorded voltage at each excitation patch of the plurality of excitation patch to the recorded voltage at the plurality of electrodes. The prediction model may also take cardiac tissue type, density and conductivity into account when performing the assessment of an impedance location. In a non-limiting embodiment, the prediction model 152 may include an impedance location machine learning model 156. By applying machine learning techniques, the software can generate a predicted impedance location accurately and quickly. Machine learning models may enable the software to learn from past collaborative experiences of the entities and iteratively improve its training data over time.

With continued reference to FIG. 1, processor 128 may be configured to update the impedance location training data of the impedance location machine-learning model using user inputs. An impedance location machine-learning model may use user input to update its training data, thereby improving its performance, speed, and accuracy. In an embodiment, impedance location training data may be configured to correlate measured voltages as inputs to impedance locations as outputs. In some embodiments, impedance location training data may be configured to correlate a voltage deviation to an impedance location. In embodiments, the impedance location machine-learning model may be iteratively updated using input and output results of past iterations of the impedance location machine-learning models. The impedance location machine-learning model 156 may then be iteratively retrained using the updated impedance location training data. For instance, and without limitation, impedance location machine-learning model may be trained using a first training data from, for example, and without limitation, training data from a user input or database. The impedance location machine-learning model 156 may then be updated by using previous inputs and outputs from the impedance location machine-learning model as second set of training data to then retrain a newer iteration of impedance location machine-learning model. This process of updating the impedance location machine-learning model and its associated training data may be continuously done to create subsequent impedance location machine-learning models to improve the speed and accuracy of the impedance location machine-learning model. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the impedance location machine-learning model and any other machine-learning model/classifier discussed herein.

Incorporating the user feedback may include updating the training data by removing or adding correlations of user data to a path or resources as indicated by the feedback. Any machine-learning model as described herein may have the training data updated based on such feedback or data gathered using any method described herein. For example, when correlations in training data are based on outdated information, a web crawler may update such correlations based on more recent resources and information.

With continued reference to FIG. 1, processor 128 may use user feedback to train the machine-learning models and/or classifiers described above. For example, machine-learning models and/or classifiers may be trained using past inputs and outputs of the machine-learning model. In some embodiments, if user feedback indicates that an output of machine-learning models and/or classifiers was “unfavorable,” then that output and the corresponding input may be removed from training data used to train machine-learning models and/or classifiers, and/or may be replaced with a value entered by, e.g., another value that represents an ideal output given the input the machine learning model originally received, permitting use in retraining, and adding to training data; in either case, machine-learning models may be retrained with modified training data as described in further detail below. In some embodiments, training data of classifier may include user feedback.

With continued reference to FIG. 1, in some embodiments, an accuracy score may be calculated for the machine-learning model and/or classifier using user feedback. For the purposes of this disclosure, “accuracy score,” is a numerical value concerning the accuracy of a machine-learning model. For example, the accuracy/quality of the output impedance location machine-learning model may be averaged to determine an accuracy score. In some embodiments, an accuracy score may be determined for pairing of entities. Accuracy score or another score as described above may indicate a degree of retraining needed for a machine-learning model and/or classifier. Processor 128 may perform a larger number of retraining cycles for a higher number (or lower number, depending on a numerical interpretation used), and/or may collect more training data for such retraining. The discussion within this paragraph and the paragraphs preceding this paragraph may apply to both the impedance location machine-learning model and/or any other machine-learning model/classifier mentioned herein.

With continued reference to FIG. 1, processor 128 may be configured to retrieve a location of the medical device 160 as a function of the impedance location. The location of the medical device may be indicated using a three-dimensional coordinate associated with a position of the medical device within the three-dimensional volume, e.g., (Mx, My, Mz). The three-dimensional coordinate may indicate a medical device's precision location within the three-dimensional volume. The location of each excitation patch of the plurality of excitation patches may also be indicated using a three-dimensional coordinate. Processor 128 may estimate the location of the medical device as a function of the voltage measurements at the plurality of electrodes. Processor 128 may be configured to integrate the measurements of the at least a voltage at the plurality of electrodes and the assessment of impedance location to predict the medical device's location. Processor 128 may be configured to analyze any differences in the voltage readings and impedance location data to estimate the medical device's position. In an embodiment, retrieving the location of the medical device 160 may include aggregating the assessment of the impedance location between each excitation patch of the plurality of excitation patches and the plurality of electrodes. Evaluating the location of the medical device based on the aggregate assessment of impedance data relative to each excitation patch within the plurality of excitation patches may enhance the accuracy of determining the device's position. By aggregating impedance measurements from multiple excitation patches, processor 128 can cross-reference and validate the spatial coordinates. This multi-faceted approach can ensure that the location of the medical device is determined with a higher degree of precision, taking into account variations in tissue characteristics and electrical conductivity.

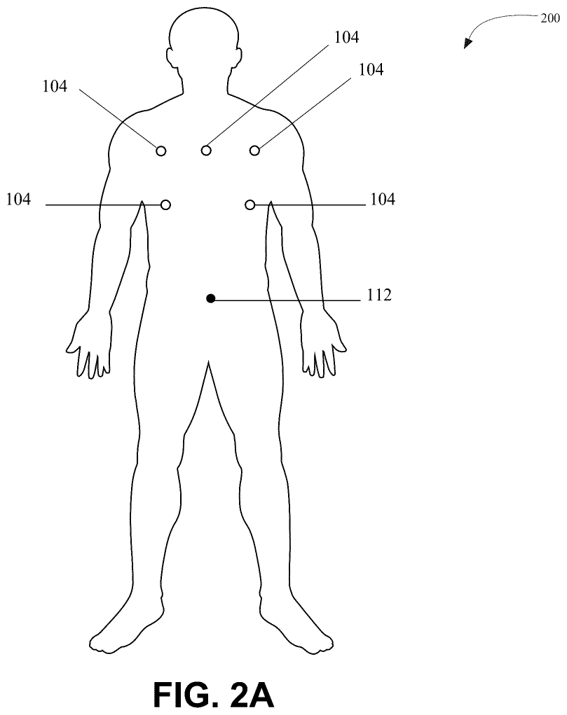

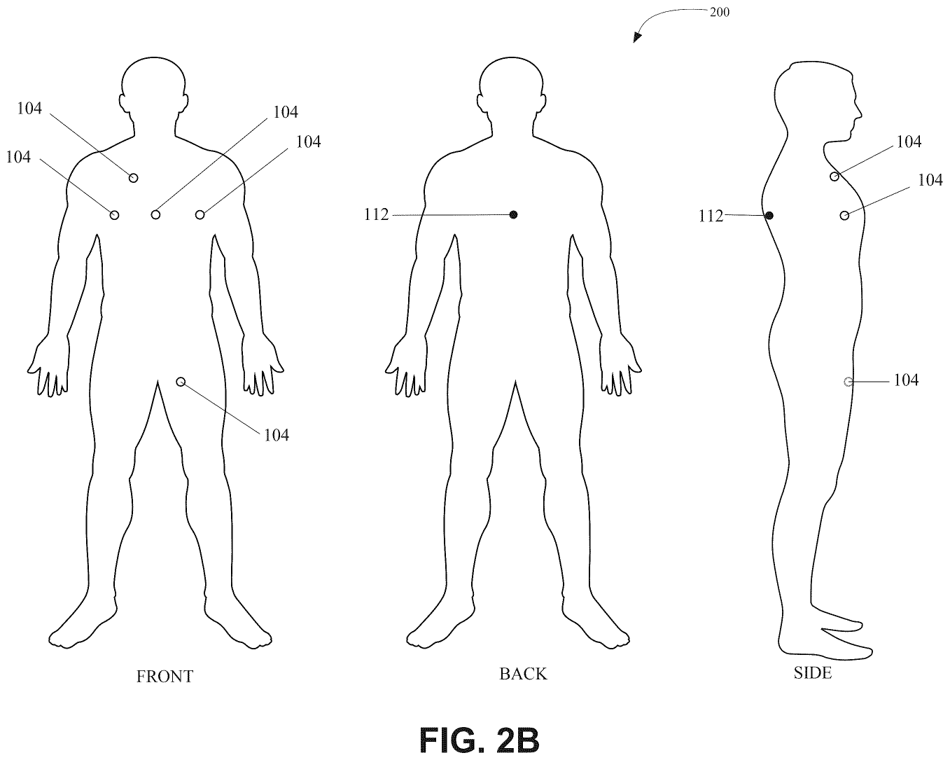

Now referencing FIGS. 2A-B, an exemplary positioning 200 of the plurality of excitation patches on a human body are shown. Elements of FIGS. 2A-B are explained in conjunction with elements of FIG. 1. In an embodiment, each excitation patch of the plurality of excitation patches may be adhered to the human body. The common ground patch may also be adhered to the human body.

Referencing FIG. 2A, in some embodiments, the plurality of excitation patches 104 may be strategically positioned on the upper portion of the human body to generate a hemispherical electrical field. This configuration may be achieved by situating the common ground patch on the lower portion of the human body. By arranging the plurality of excitation patches in this manner, the system may create an optimal field distribution, ensuring comprehensive coverage and enhanced signal transmission. This strategic placement leverages the human body's structure to facilitate a hemispherical electrical field formation. The hemispherical field generated through this arrangement may maximize the efficiency of the plurality of excitation patches and the common ground patch, which can lead to lower probability of a dead zone within the electric field.

Referencing FIG. 2B, in some embodiments, the plurality of excitation patches 104 may be positioned across various points on the front of the human body, while the common ground patch 112 may be located on the back of the human body. This configuration may enable the generation of a hemispherical electrical field. In some embodiments, arranging the excitation patches on the front and the common ground patch on the back, the system may achieve an optimal distribution of the electrical field. The association between the front-placed excitation patches and the back-placed common ground patch may maximize the system's efficiency, promoting better overall performance and interaction within the electrical field.

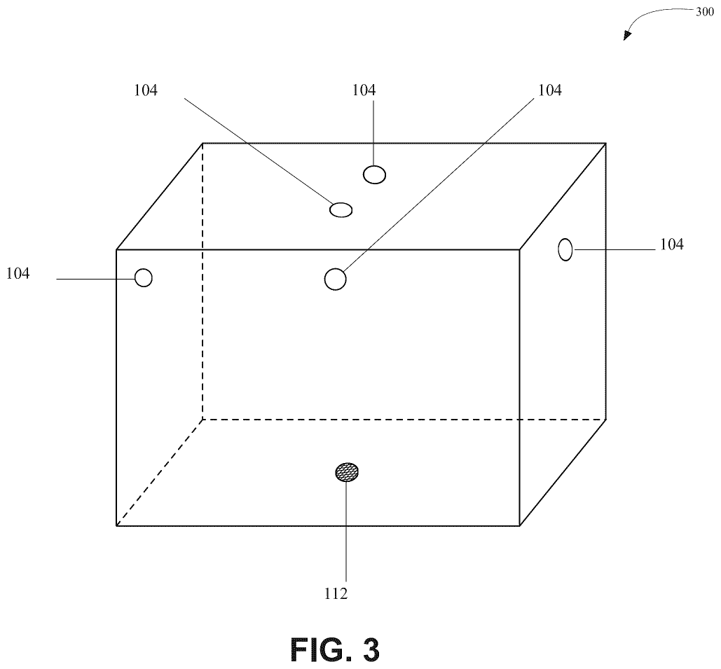

Now referencing FIG. 3, exemplary positioning 300 of the plurality of excitation patches on a three-dimensional volume is illustrated. In a non-limiting embodiment, each excitation patch of the plurality of excitation patches is placed significantly near to the top face of the three-dimensional volume. In another embodiment, there may be multiple additional excitation patches. In a non-limiting embodiment, this configuration of the plurality of excitation patches 104 may be used in conjunction with time multiplexing for the excitation as used in existing EP mapping solutions.

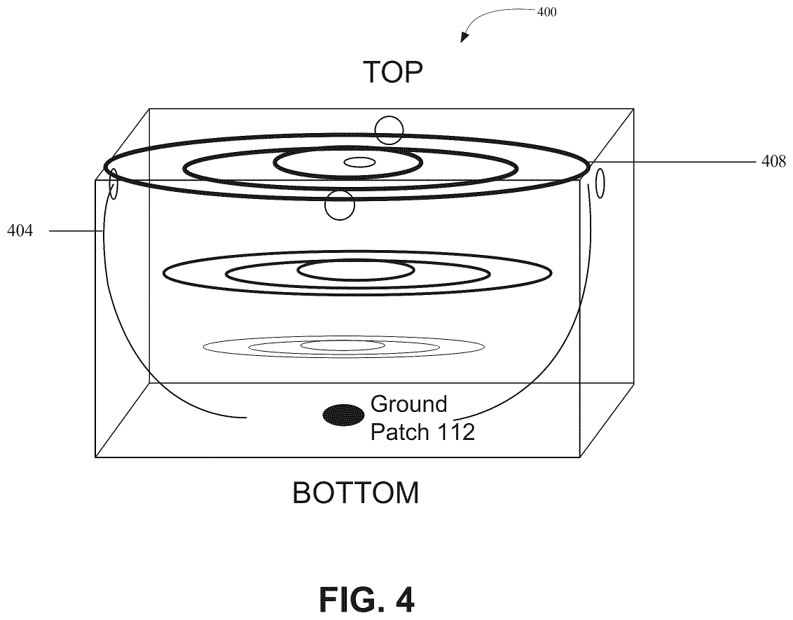

Now referring FIG. 4, an exemplary embodiment 400 of a hemispherical field within a three-dimensional volume is illustrated. In an embodiment, curve 404 represents an isopotential line. The isopotential line indicates that the potential energy of a charged particle remains constant at any location along curve 404. Field 408 represents the electric field produced by the plurality of excitation patches 104 and the common ground patch 112.

Referring now to FIG. 5, illustrations of an exemplary embodiment of a catheter assembly 500 is disclosed. As used in the current disclosure, a “catheter” is a flexible, tube-like medical device used to perform various diagnostic and therapeutic procedures within the body. A catheter assembly 500 may be made from medical-grade materials such as silicone, rubber, or polyurethane, allowing it to navigate through the vascular system, urinary tract, or other body cavities. Catheter assemblies 500 may vary in size, length, and tip configuration, tailored to specific medical applications, such as delivering medications, draining fluids, or performing complex surgical tasks like cardiac ablation and mapping. In cardiology, catheter assemblies 500 may be used for procedures such as angiography, where they inject contrast dye into the heart vessels for imaging, or for electrophysiology studies and interventions that involve mapping electrical activity and ablating faulty electrical pathways in the heart. Their design often includes features like radio-opaque materials, which make them visible under X-ray guidance during procedures, enhancing safety and precision. In an embodiment, a catheter assembly 500 may include a plurality of components. These components may include but are not limited to an ablation tool, catheter tip, catheter shaft, hub, guidewire, guidewire lumen, markers, control mechanisms, coatings, and the like.

With continued reference to FIG. 5, a catheter assembly 500 may include a tip 504. As used in the current disclosure, a “catheter tip” is the distal end of the catheter. A catheter tip 504 may be designed to interact directly with body tissues or systems during medical procedures. Tips 504 may vary significantly in structure and functionality depending on its specific medical application. For example, in cardiac catheterization, the tip may be engineered to conduct electrical signals for diagnostic measurements or to deliver energy for ablation therapy. In contrast, tips used in intravenous catheters are optimized for smooth insertion and minimal discomfort, often featuring a tapered or beveled design. Catheter tips can also include specialized features such as ultrasound transducers for imaging, balloons for dilating vessels or valves, or baskets for retrieving stones or debris from bodily organs. Additionally, many catheter tips 504 may include sensors that provide real-time feedback on physiological parameters or the precise positioning of the tip within the body. The design of the catheter tip is critical to the success of the procedure, influencing factors such as ease of navigation through the body's pathways, the effectiveness of the treatment delivered, and the overall safety of the procedure.

With continued reference to FIG. 5, a catheter assembly 500 includes a plurality of electrodes 508 on the tip 504. As used in the current disclosure, an “electrodes” is a multi-functional component that is used for both diagnostic and therapeutic functions in medical procedures. Located on the tip 504 of the catheter, each electrode 508 may be engineered to perform critical functions essential for the successful execution of catheter-based interventions. The electrodes 508 may include conductive components used for therapy, such as delivering targeted treatments like ablation within the body, but they also serve diagnostic purposes. This dual functionality is integral to procedures like cardiac ablation, where precise location and treatment of aberrant electrical pathways are crucial. The therapeutic aspect typically involves the application of energy—be it radiofrequency, cryogenic, or others—to modify or destroy tissue responsible for abnormal activity, effectively treating conditions such as arrhythmias. In an embodiment, each electrode 508 may include a biomedical sensor. This biomedical sensor may enable real-time physiological monitoring. These sensors can measure various bioelectrical or biochemical parameters, providing continuous feedback on the local environment at the tip of the catheter. For instance, in cardiac applications, these sensors might record electrical signals from the heart tissue, aiding in the detailed mapping of electrical activity. This mapping is crucial for identifying precise treatment sites and for monitoring the effects of therapy, ensuring both the efficacy and safety of the procedure.

With continued reference to FIG. 5, each electrode 508 may include a biomedical sensor. As used in the current disclosure, a “biomedical sensor” is a specialized component that enhances the functionality of the electrode by enabling it to perform detailed physiological measurements directly from the site of interest within the body. The biomedical sensor embedded within electrode 508 is designed to detect and measure specific physiological parameters. These might include electrical activity, such as the heart's electrocardiographic signals in cardiac procedures, temperature changes, pH levels, or other biochemical markers that are critical in assessing the health and condition of tissues. In the case of cardiac applications, such sensors are crucial for accurately mapping electrical pathways and identifying arrhythmic zones. The inclusion of a biomedical sensor in the electrode 508 may enable real-time, site-specific data collection. This capability allows for immediate feedback during procedures, aiding physicians in making informed decisions based on the latest physiological data. For instance, during a cardiac ablation procedure, sensors can measure the electrical activity before, during, and after ablation, providing essential information on the effectiveness of the treatment and the stability of the tissue post-ablation. The integration of the sensor with the electrode is a critical aspect of the device's design. It ensures that while the electrode delivers therapeutic treatments like ablation, it simultaneously gathers vital diagnostic data. This integration facilitates a comprehensive approach to treatment, where therapy is continuously guided and adjusted based on direct feedback from the target site.

With continued reference to FIG. 5, the biomedical sensor may be configured to measure the impedance within the heart. Measuring impedance within the heart may provide data that can help identify arrhythmic areas and guide therapeutic interventions such as ablation. Impedance measurement may refer to evaluating the resistance and reactance of heart tissue to an electrical current. By applying a small, known current through the electrodes and measuring the resulting voltage drop, the impedance can be calculated. This measurement may provide insight into the composition and health of the tissue, as different types of tissue (healthy, diseased, scarred) have distinct impedance characteristics. The biomedical sensor integrated into electrode 124 may include built-in impedance measurement capabilities. This may be used to perform real-time monitoring during procedures. These sensors may be designed to be highly sensitive and can differentiate between various types of cardiac tissue based on their electrical properties. In a non-limiting example, healthy myocardial tissue may reflect different impedance profile compared to scarred or fibrotic tissue, which might be targeted during ablation for arrhythmia treatment. In an embodiment, impedance location may be measured as a function of a measured voltage at electrodes 508. Additionally, blood and fluid-filled areas may also display unique impedance characteristics, which can help in avoiding or targeting specific zones during interventions. During cardiac mapping procedures, impedance measurements help create a detailed map of the heart's electrical activity and tissue health. This map is crucial for identifying the pathways that cause arrhythmias. During ablation, continuous impedance monitoring can inform the physician about the extent of tissue modification. Changes in impedance can indicate effective ablation, helping to prevent overtreatment or undertreatment. Post-ablation, impedance measurements can help assess whether the targeted tissue has been adequately modified or needs further intervention.

With continued reference to FIG. 5, the plurality of electrodes 508 on the tip 504 of the catheter assembly 500 may be used as an ablation tool 512. As used in the current disclosure, an “ablation tool” is a medical device designed for precise, targeted therapeutic interventions within the body, particularly in treating cardiac arrhythmias such as atrial fibrillation. This tool may incorporate multiple electrodes 508 on the catheter's tip 504. Each electrode 508 may be capable of delivering energy to specific areas of tissue to modify or destroy it selectively. In an embodiment, each electrode 508 in the plurality can be individually controlled to emit specific types and amounts of energy, such as radiofrequency, cryoenergy, or laser, allowing for versatile treatment strategies. The ability to control each electrode independently helps in tailoring the ablation precisely to the size, shape, and location of the target tissue, enhancing the efficacy of the procedure while minimizing damage to surrounding healthy tissues.

With continued reference to FIG. 5, an ablation tool 512 composed of a plurality of electrodes may be used to treat cardiac arrhythmias such as atrial fibrillation. This tool may incorporate multiple electrodes 124 at its tip, each capable of delivering energy to specific areas of tissue to modify or destroy it selectively. Here's a detailed description of such an ablation tool: The electrodes may be arranged at the distal end of the catheter assembly 500. The electrodes 508 may be arranged in a circular, linear, or array format depending on the specific medical requirement. These electrodes 508 may be made from conductive materials such as platinum or gold, which are chosen for their durability, conductivity, and biocompatibility. The arrangement of the electrodes is crucial as it allows for the delivery of ablation energy in a controlled and focused manner. In an embodiment, each electrode 508 within the plurality can be individually controlled to emit specific types and amounts of energy, such as radiofrequency, cryoenergy, or laser, allowing for versatile treatment strategies. The ability to control each electrode independently may allow for precision ablation tailored to the size, shape, and location of the target tissue, enhancing the efficacy of the procedure while minimizing damage to surrounding healthy tissues.

With continued reference to FIG. 5, the arrangement of electrodes 508 on catheter assembly 500 may play a role in delivering targeted therapeutic interventions and collecting detailed diagnostic data. Electrodes can be arranged in various configurations to suit specific medical requirements, enhancing the precision and effectiveness of the catheter. Electrodes 508 on a catheter assembly 500 can be arranged in several patterns, including linear, circular, rows, or in a grid. Each arrangement may be designed to maximize the catheter's ability to interact with the tissue it targets, depending on the procedure's needs. The plurality of electrodes 508 may be arranged in a grid arrangement. As used in the current disclosure, a “grid arrangement” is an arrangement of electrodes 508 in rows and columns. A grid arrangement may include a plurality of rows and columns each containing multiple electrodes 508. In a non-limiting example, a grid arrangement of the plurality of electrodes could be in three rows of four electrodes 508. This specific configuration may allow for a broad and yet precise area of coverage, making it ideal for procedures that require a nuanced approach to tissue interaction, such as cardiac ablation. By arranging the electrodes in multiple rows, the catheter can cover a larger surface area while maintaining the ability to target specific tissue sections accurately. This is particularly useful in cardiology, where large areas of the heart may need to be mapped and treated for arrhythmias. The configuration of the plurality of electrodes 508 may allow detailed electrical mapping of the heart's activity. The grid arrangement may enable the catheter to collect data from multiple points simultaneously, providing a comprehensive map of electrical signals across different layers and sections of heart tissue. In therapeutic applications, such as ablation, a grid arrangement may allow for segmented or selective energy delivery. For instance, only one or two rows of electrodes might be activated at a time, depending on the specific area requiring ablation. This capability ensures that the ablation is confined to the intended tissue, minimizing the impact on adjacent areas.

With continued reference to FIG. 5, the arrangement of electrodes 508 on catheter assembly 500 includes a plurality of constraint pairs 516. As used in the current disclosure, “constraint pairs” refer to predefined relationships between pairs of electrodes that must remain constant or within certain limits during the use of the catheter. These pairs 516 may ensure that the catheter's configuration adheres to specific design and functional requirements, especially during complex medical procedures such as cardiac mapping or ablation. Constraint pairs 516 may be used to define the fixed or controlled distances and orientations between specific electrodes on a catheter. Constraint pairs 516 may include a rigid position of the tip 504 that separates each electrode 508 of the plurality of electrodes. These constraint pairs 516 may be used to maintain the structural integrity and functional capability of the catheter as it navigates through the body. Constraint pairs 516 may ensure that the catheter operates within its designed specifications. This compliance is vital for device certification and regulatory approval, as well as for ensuring that the device performs as tested during clinical trials. In medical applications, maintaining these constraints ensures that the catheter can perform its intended functions accurately. Constraint pairs 516 may help maintain the physical stability of the catheter by ensuring that the electrodes do not move into configurations that could be physically impossible or harmful to the patient. For example, they prevent electrodes from collapsing into each other or stretching too far apart, which could compromise the catheter's effectiveness or cause damage to body tissues. In procedures that rely on the precise application of energy (such as ablation) or the accurate measurement of electrical signals (such as impedance mapping), constraint pairs ensure that the spatial relationships necessary for accurate readings and energy delivery are maintained. This accuracy is crucial for the efficacy of the treatment and the safety of the procedure. In a non-limiting example, suppose a catheter has multiple electrodes arranged along its length intended for cardiac ablation. The constraint pairs 516 may specify that certain electrodes 508 must remain within a specific distance from each other to ensure optimal energy delivery to the heart tissue. If the catheter is designed to create a circular lesion, the constraint pairs might specify the angles and distances necessary to form a perfect circle, ensuring that all parts of the targeted area receive equal treatment.

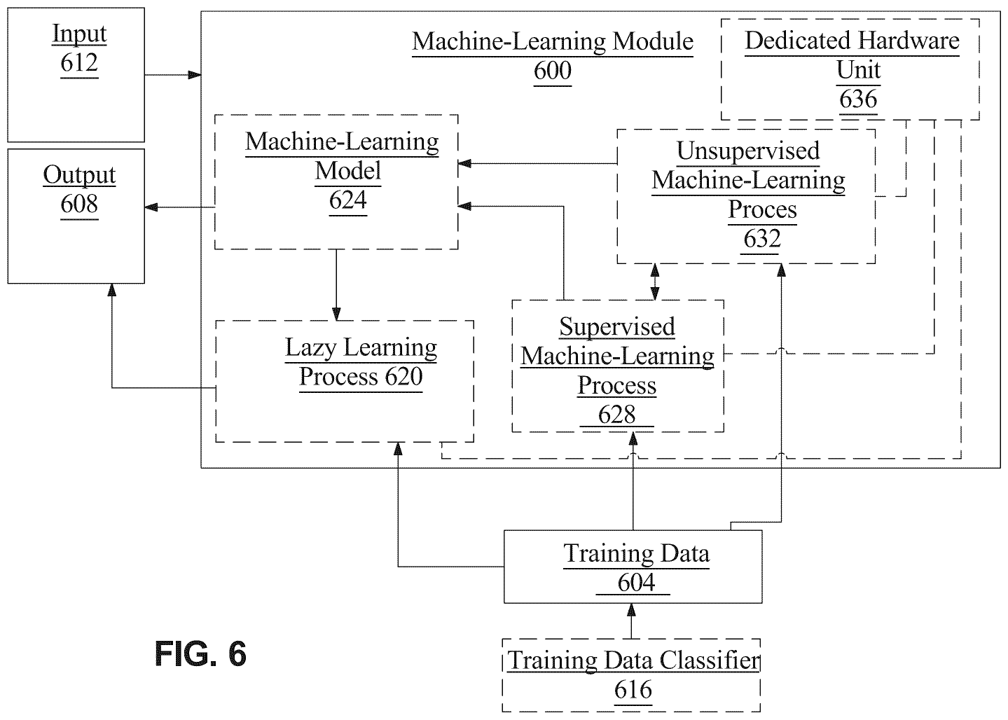

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm instantiated in hardware or software logic, data structures, and/or functions that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure. As a non-limiting illustrative example, exemplary training data may include a plurality of first impedance locations as input correlated with a plurality of magnetic locations as output.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 6, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 6, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 6, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 6, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 6, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 6, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 6, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 6, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 6, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 6, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 6, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{ma x} : {X}_{n e w} = \frac{X - {X}_{m i n}}{{X}_{m ax} - {X}_{m i n}} .$$

Feature scaling may include mean normalization, which involves use of a mean value of a set and/or subset of values, Xₘₑₐₙ with maximum and minimum values:

$${X}_{n e w} = \frac{X - {X}_{m ean}}{{X}_{m ax} - {X}_{m i n}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 6, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include plurality of first impedance locations as described above as inputs, a plurality of magnetic locations as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

With further reference to FIG. 6, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 6, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 632 may not require a response variable; unsupervised processes 632 may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 6, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 6, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 6, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 6, one or more processes or algorithms described above may be performed by at least a dedicated hardware unit 636. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 636 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 636 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 636 to perform one or more operations described herein, such as evaluation of model and/or algorithm outputs, one-time or iterative updates to parameters, coefficients, weights, and/or biases, and/or any other operations such as vector and/or matrix operations as described in this disclosure.

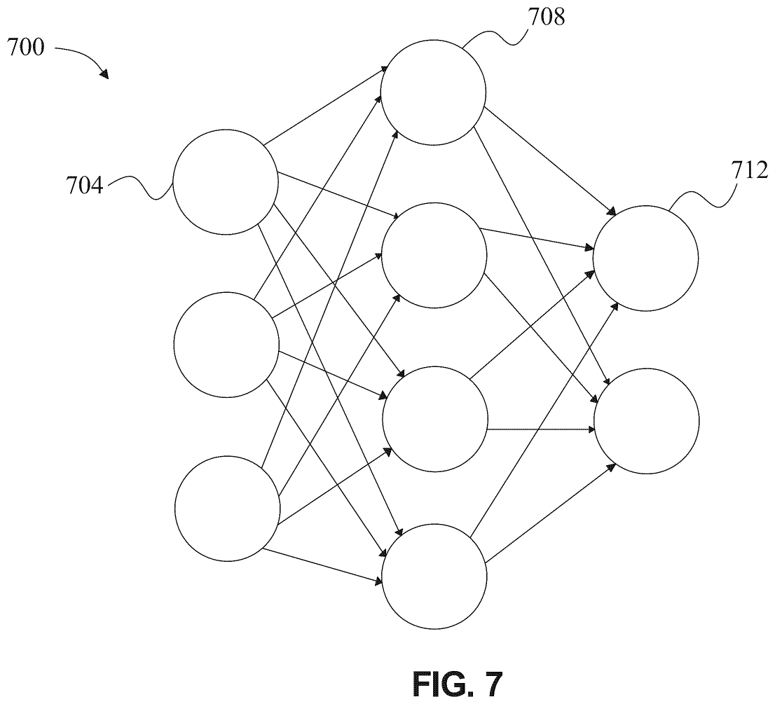

Referring now to FIG. 7, an exemplary embodiment of neural network 700 is illustrated. In some cases, prediction model 152 may include a neural network 700. Neural network 700 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 704, one or more intermediate layers 708, and an output layer of nodes 712. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network”.

With continued reference to FIG. 7, as a further non-limiting example, a neural network may include a convolutional neural network comprising an input layer of nodes, one or more intermediate layers, and an output layer of nodes. A “convolutional neural network,” for the purpose of this disclosure, is a neural network in which at least one hidden layer is a convolutional layer that convolves inputs to that layer with a subset of inputs known as a “kernel,” along with one or more additional layers such as pooling layers, fully connected layers, and the like. In some cases, CNN may include, without limitation, a deep neural network (DNN) extension. Mathematical (or convolution) operations performed in the convolutional layer may include convolution of two or more functions, where the kernel may be applied to input data through a sliding window approach. In some cases, convolution operations may enable processor 128 to detect local/global patterns, edges, and any other features described herein within training data. Detected patterns and/or features may be passed through one or more activation functions, such as without limitation, Rectified Linear Unit (ReLU). Additionally, or alternatively, CNN may also include one or more pooling layers, wherein each pooling layer is configured to reduce the dimensionality of input data while preserving essential features within the input data. In a non-limiting example, CNN may include one or more pooling layer configured to reduce the dimensions of feature maps by applying downsampling, such as max-pooling or average pooling, to small, non-overlapping regions of one or more features. CNN may further include one or more fully connected layers configured to combine features extracted by the convolutional and pooling layers as described above. In some cases, one or more fully connected layers may allow for higher-level pattern recognition. In a non-limiting example, one or more fully connected layers may connect every neuron (i.e., node) in its input to every neuron in its output, functioning as a traditional feedforward neural network layer. In some cases, one or more fully connected layers may be used at the end of CNN to perform high-level reasoning and produce the final output such as, without limitation, at least one updated second impedance location. Additionally, or alternatively, each fully connected layer may be followed by one or more dropout layers configured to prevent overfitting, and one or more normalization layers to stabilize the learning process described herein.

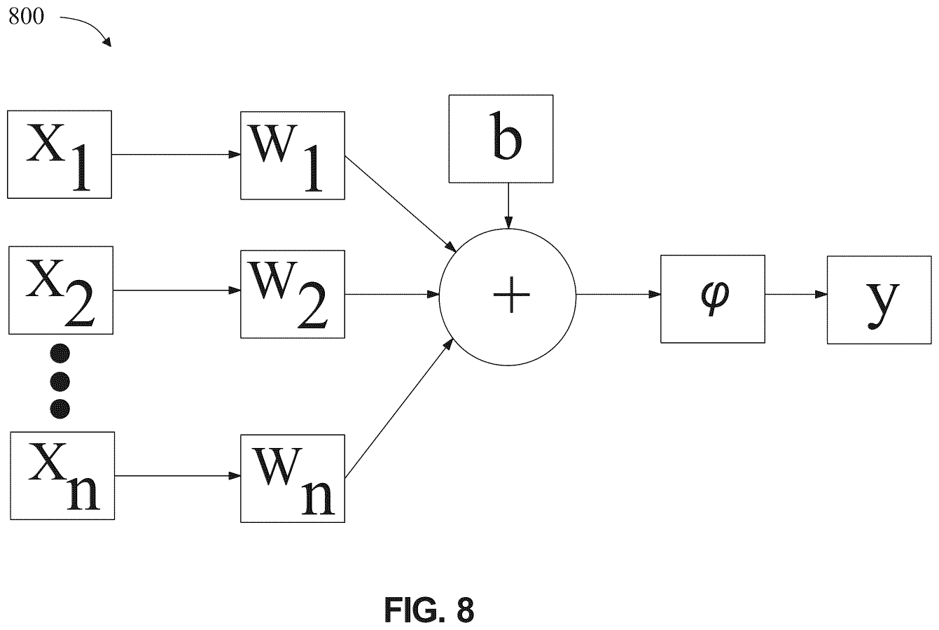

Referring now to FIG. 8, an exemplary embodiment of a node 800 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0,x), a “leaky” and/or “parametric” rectified linear unit function such as ƒ(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of a (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as ƒ(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs x; that may be used as activation functions. As a non-limiting and illustrative example, node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

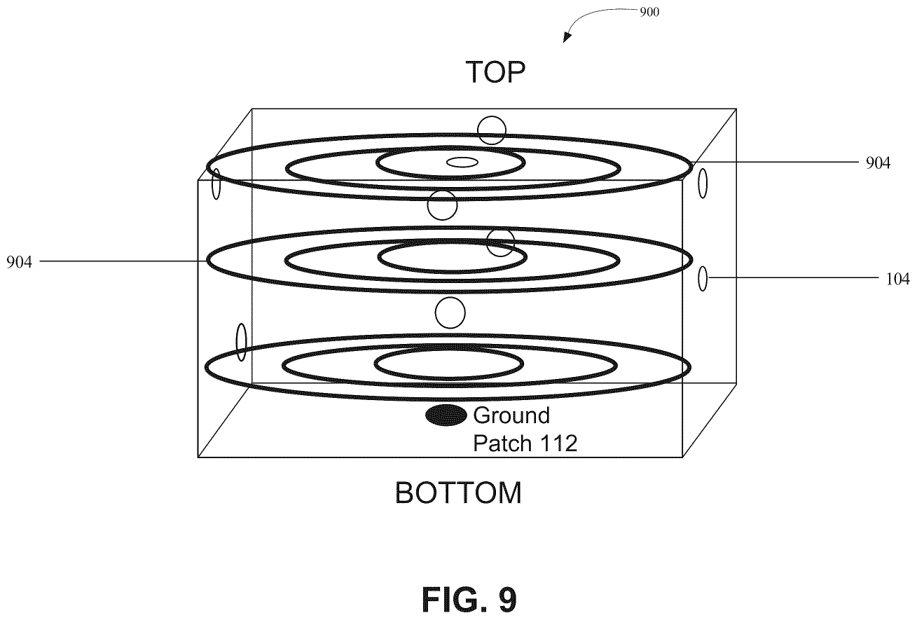

Now referring to FIG. 9, an exemplary embodiment of a cylindrical field is illustrated. In an embodiment, the hemispherical field 108 is configured to transform into a cylindrical field 904 as a function of excitation patch 104 configuration wherein the positioning of at least an additional excitation patch transforms the hemispherical field to a cylindrical field. In an embodiment, placement of an additional excitation patch may alter the electromagnetic properties of the hemispherical field, thereby reshaping the hemispherical field into a cylindrical shape.

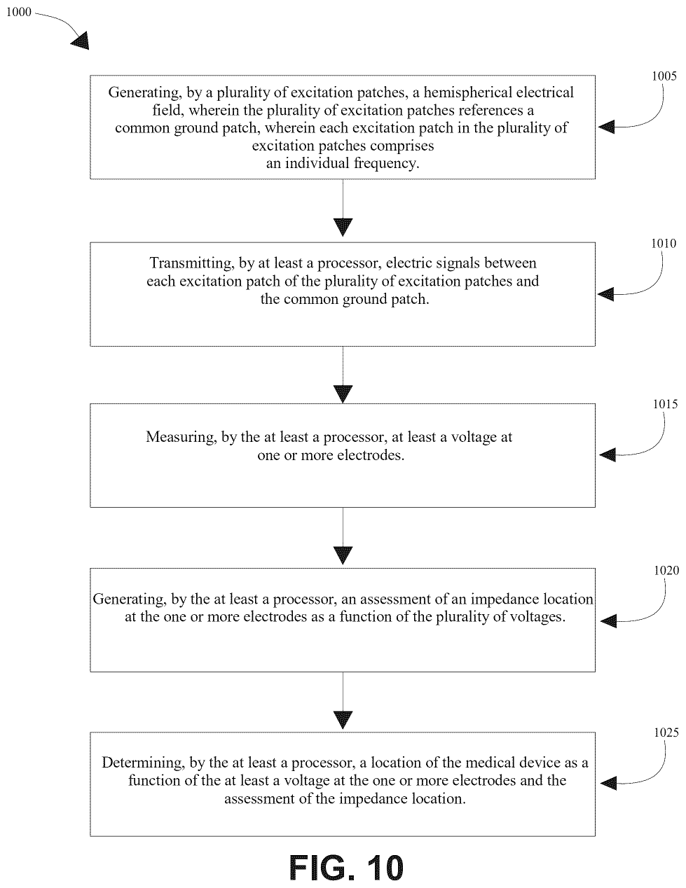

Now referring to FIG. 10, a flow diagram for an exemplary embodiment of a method 1000 for locating a medical device using an electrical field creation is illustrated. Method 1000 includes a step 1005 of generating, by a plurality of excitation patches, a hemispherical electrical field, wherein the plurality of excitation patches references a common ground patch, wherein each excitation patch in the plurality of excitation patches comprises an individual frequency. In an embodiment, the hemispherical field is configured to transform into a cylindrical field as a function of excitation patch configuration wherein the positioning of at least an additional excitation patch transforms the hemispherical field to a cylindrical field. This may be implemented without limitation as described above with reference to FIS. 1-8.

Continuing reference to FIG. 10, Method 1000 includes a step 1010 of transmitting, by at least a processor, an electric signal between each excitation patch of the plurality of excitation patches and the common patch. In some embodiments, transmitting the electrical signal between each excitation patch of the plurality of excitation patches and the common patch includes transmitting an alternating current. In some embodiments, transmitting the electrical signal between each excitation patch of the plurality of excitation patches and the common patch includes sequentially transmitting the electrical signal from each excitation patch of the plurality of excitation patches. This may be implemented without limitation as described above with reference to FIS. 1-8.

Continuing reference to FIG. 10, method 1000 includes a step 1015 of measuring, by at least a processor at least a voltage at one or more of electrodes. This may be implemented, without limitation as described above with reference to FIS. 1-8.

Continuing reference to FIG. 10, method 1000 includes a step 1020 of generating, by the at least a processor, an assessment of an impedance location at the one more electrode as a function of the plurality of voltages. In some embodiments, generating an assessment of an impedance location at the one or more electrodes includes a prediction model configured to measure the voltage deviation of each excitation patch of the plurality of excitation patches to the plurality of electrodes and convert the voltage deviation to the impedance location for the plurality of electrodes. In some embodiments, the prediction model includes an impedance location machine learning model iteratively trained using training data configured to correlate predicted voltage deviation inputs to predicted impedance location outputs. In some embodiments, assessment of the impedance location is calibrated as a function of movement of the three-dimensional volume. This may be implemented, without limitation as described above with reference to FIS. 1-8.

Continuing reference to FIG. 10, method 1000 includes a step 1025 of determining, by the at least a processor, a location of the medical device as a function of the at least a voltage at the one or more of electrodes and the assessment of the impedance location. In some embodiments, retrieving the location of the medical device includes aggregating the assessment of the impedance location between each excitation patch of the plurality of excitation patches and the plurality of electrodes. This may be implemented, without limitation as described above with reference to FIS. 1-8.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

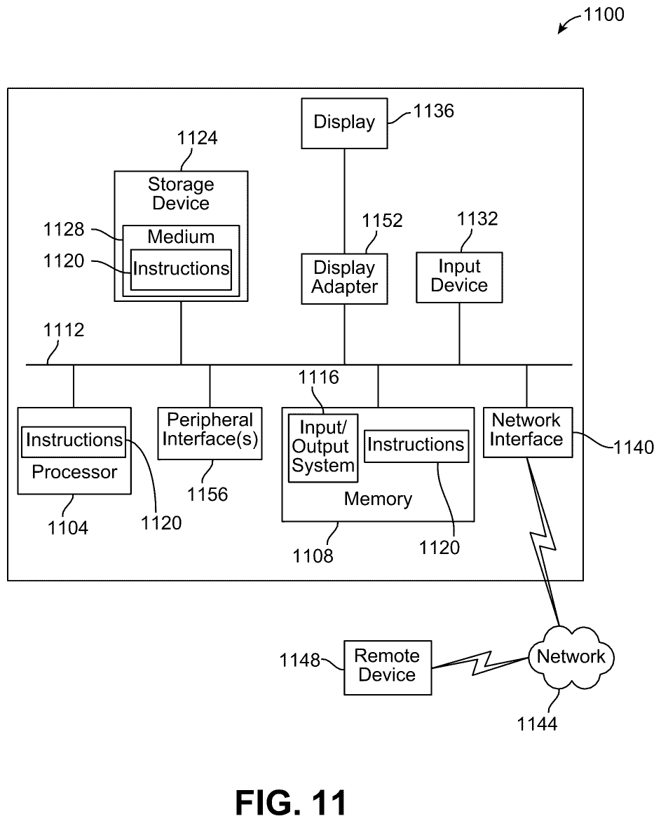

FIG. 11 shows a diagrammatic representation of one embodiment of a computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display device 1136. Examples of a display device include, but are not limited to, a liquid crystal display (LCD), a cathode ray tube (CRT), a plasma display, a light emitting diode (LED) display, and any combinations thereof. Display adapter 1152 and display device 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for locating a medical device using an electrical field creation, the system comprising:
a plurality of excitation patches configured to generate a hemispherical electrical field, wherein the plurality of excitation patches references a common ground patch, wherein each excitation patch in the plurality of excitation patches comprises an individual frequency;
at least a catheter assembly comprising:
at least a tip, wherein the at least a tip is comprised of:
one or more electrodes, including at least a biomedical sensor embedded within each electrode of the one or more electrodes and configured detect and measure physiological parameters including an impedance measurement; and
a plurality of constraint pairs configured to maintain a predefined relationships between pairs of electrodes for structural integrity and functional capability of the catheter assembly;
at least a processor communicatively connected to the plurality of excitation patches and the at least a catheter assembly; and
a memory communicatively connected to the at least a processor, wherein the memory contains instructions configuring the at least a processor to:
transmit electrical signals between each excitation patch of the plurality of excitation patches and the common ground patch;
measure at least a voltage at the one or more electrodes;
generate an assessment of an impedance location at an electrode of the one or more electrodes as a function of a plurality of voltages; and
determine a location of the medical device as a function of the at least a voltage at the one or more electrodes and the assessment of the impedance location further comprising aggregating the assessment of the impedance location between each excitation patch of the plurality of excitation patches and the plurality of electrodes, wherein the at least a processor additionally cross-references and validates spatial coordinates related to the plurality of excitation patches to enhance accuracy of determining the location of the medical device to perform at least a cardiac ablation.

2. The system of claim 1, wherein the electrical signals transmitted between each excitation patch of the plurality of excitation patches and the common ground patch is an alternating current.

3. The system of claim 1, wherein transmitting the electrical signals between each excitation patch of the plurality of excitation patches comprises sequentially transmitting the electrical signals from each excitation patch of the plurality of excitation patches.

4. The system of claim 1, wherein generating the assessment of the impedance location comprises a prediction model.

5. The system of claim 4, wherein the prediction model is configured to:
determine a relative distance to each excitation patch of the plurality of excitation patches as a function of the measured at least a voltage at the one or more electrodes; and
convert the relative distance to absolute coordinates using a known position of each excitation patch of the plurality of excitation patches.

6. The system of claim 4, wherein the prediction model comprises an impedance location machine learning model iteratively trained using training data configured to correlate voltage deviation inputs to impedance location outputs.

7. The system of claim 1, wherein the configuration of the plurality of excitation patches eliminates dead zones in an electric field created by the electrical signals.

8. The system of claim 1, wherein retrieving the location of the medical device comprises creating an assessment of the impedance location between excitation patch of the plurality of excitation patches and the one or more electrodes, and aggregating the assessment of the impedance location between each excitation patch of the plurality of excitation patches and the one or more electrodes.

9. The system of claim 1, wherein the memory contains instructions further configuring the at least a processor to calibrate the assessment of the impedance location as a function of a movement of one of more of the plurality of excitation patches.

10. The system of claim 1, wherein the hemispherical electrical field is configured to transform into a cylindrical field as a function of excitation patch configuration wherein a positioning of at least an additional excitation patch transforms the hemispherical field to a cylindrical field.

11. A method of locating a medical device using an electrical field creation, the method comprising:
generating, by a plurality of excitation patches, a hemispherical electrical field, wherein the plurality of excitation patches references a common ground patch, wherein each excitation patch in the plurality of excitation patches comprises an individual frequency;
transmitting, by at least a processor, electrical signals between each excitation patch of the plurality of excitation patches and the common ground patch;
measuring, by the at least a processor, at least a voltage at one or more electrodes of a catheter assembly comprising at least a tip wherein the at least a tip is comprised of:
at least a biomedical sensor embedded within each electrode of the one or more electrodes and configured detect and measure physiological parameters including an impedance measurement; and
a plurality of constraint pairs configured to maintain a predefined relationships between pairs of electrodes for structural integrity and functional capability of the catheter assembly;
generating, by the at least a processor, an assessment of an impedance location at the one or more electrodes as a function of a plurality of voltages; and
determining, by the at least a processor, a location of the medical device as a function of the at least a voltage at the one or more electrodes and the assessment of the impedance location further comprising aggregating the assessment of the impedance location between each excitation patch of the plurality of excitation patches and the plurality of electrodes, wherein the at least a processor additionally cross-references and validates spatial coordinates related to the plurality of excitation patches to enhance accuracy of determining the location of the medical device to perform at least a cardiac ablation.

12. The method of claim 11, wherein the electrical signals transmitted between each excitation patch of the plurality of excitation patches and the common ground patch is an alternating current.

13. The method of claim 11, wherein transmitting the electrical signals between each excitation patch of the plurality of excitation patches comprises sequentially transmitting the electrical signals from each excitation patch of the plurality of excitation patches.

14. The method of claim 11, wherein generating the assessment of the impedance location comprises a prediction model.

15. The method of claim 14, wherein the prediction model is configured to:
determine a relative distance to each excitation patch of the plurality of excitation patches as a function of the measured at least a voltage at the one or more electrodes; and
convert the relative distance to absolute coordinates using a known position of each excitation patch of the plurality of excitation patches.

16. The method of claim 14, wherein the prediction model comprises an impedance location machine learning model iteratively trained using training data configured to correlate voltage deviation inputs to predicted impedance location outputs.

17. The method of claim 11, wherein the configuration of excitation patches eliminates dead zones in an electric field created by the electrical signals.

18. The method of claim 11, wherein retrieving the location of the medical device comprises creating an assessment of the impedance location between excitation patch of the plurality of excitation patches and the one or more electrodes, and aggregating the assessment of the impedance location between each excitation patch of the plurality of excitation patches and the plurality of electrodes.

19. The method of claim 11, further comprising calibrating, using the at least a processor, the assessment of the impedance location as a function of a movement of one of more of the plurality of excitation patches.

20. The method of claim 11, wherein the hemispherical electrical field is configured to transform into a cylindrical field as a function of excitation patch configuration wherein a positioning of at least an additional excitation patch transforms the hemispherical field to a cylindrical field.
